(19) United States
(12) Patent Application Publication
(10) Pub. No.: US 2008/0009633 A1
(43) Pub. Date: Jan. 10, 2008

(54) Method for Producing High-Purity Quaternary Ammonium Compounds

(75) Inventors: Laszlo Szarvas, Ludwigshafen (DE); Klemens Massonne, Bad Durkheim (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 11/661,020

(22) PCT Filed: Aug. 3, 2005
(86) PCT No.: PCT/EP05/08411

§ 371 Date: Feb. 23, 2007

(30) Foreign Application Priority Data
Aug. 24, 2004 (DE) 102004041126.3

(51) Int. Cl. C07D 213/20; C07C 209/20; C07D 233/54

(52) U.S. Cl. 546/347; 548/335.1; 564/281

(57) ABSTRACT

Process for preparing quaternary ammonium compounds by reacting the corresponding tertiary sp³-hybridized amine or sp²-hybridized imine with dimethyl sulfite, wherein the reaction is carried out
(i) in the presence of an inorganic or organic protic acid having a pKₐ of from 1.8 to 14, measured at 25° C. in aqueous solution; and (ii) at a temperature of from 10 to 100° C.

The present invention relates to a process for preparing quaternary ammonium compounds by reacting the corresponding tertiary sp³-hybridized amine or sp²-hybridized imine with dimethyl sulfite.

Quaternary ammonium compounds are important substances which are used in a wide variety of applications. Thus, they are used, for example, as active ingredients in laundry softeners, in personal hygiene products and cosmetics, as phase transfer catalysts or as electrolyte salts for electronic applications. A further important application area is ionic liquids having alkylammonium, imidazolium or pyridinium as cations.

Quaternary ammonium compounds having at least one methyl group on the nitrogen and a freely selectable anion are usually prepared in a two-step reaction. In the first step of the synthesis, the corresponding tertiary amine/imine is methylated by means of a methylating agent, with the anion of the quaternary ammonium compound obtained being determined by the methylating agent used. To introduce the desired anion, an anion exchange is subsequently carried out in the second step of the synthesis.

The methylation (first step of the synthesis) is usually carried out by reacting the corresponding tertiary amines/imines with methylating agents.

Methylating agents customarily used are the methyl esters of strong mineral acids, in particular dimethyl sulfate or methyl chloride (cf., for example, Houben-Weyl, Methoden der organischen Chemie, 4th edition, volume XI/2, Georg Thieme Verlag, Stuttgart 1958, pages 591 to 630). A disadvantage of the use of dimethyl sulfate is its carcinogenic action, which represents a hazard potential and requires elaborate safety measures. Disadvantages of the use of methyl chloride are its relatively low reactivity and consequently a relatively high reaction temperature and also a relatively high reaction pressure. This results in secondary reactions which make the work-up more difficult and reduce the yield.

As an alternative, the use of dimethyl carbonate as methylating agent is described in JP 04-341,593 and JP 09-025,173. Disadvantages of this are its relatively low reactivity and consequently a relatively high reaction temperature of over 100° C. and also a relatively high reaction pressure of from about 1 to 4 MPa abs. This results in secondary reactions which make the work-up more difficult and reduce the yield. Thus, for example, when imidazole is methylated under these conditions, carboxylation of the ring occurs. When tertiary alkylamines are used as starting materials, the Hoffmann degradation takes place under these conditions.

Furthermore, methyl iodide is also known as methylating agent for the preparation of quaternary ammonium compounds. However, a disadvantage of the use of methyl iodide is its carcinogenic action which represents a hazard potential and requires elaborate safety measures. Furthermore, methyl iodide is not available in the required industrial amounts or is relatively expensive compared to the abovementioned methylating agents.

The use of dimethyl sulfite as methylating agent for the preparation of quaternary ammonium compounds is also known per se. Thus, the DE patent 228 247 describes the reaction of various alkaloids of the morphine group with dimethyl sulfite in the presence of methanol as solvent by heating on a water bath to form the corresponding morphinium methylsulfites (described as “methylatesulfites” in the old nomenclature used in the DE text). Isolation of the morphinium methylsulfites was carried out by distilling off the solvent and excess dimethyl sulfite under reduced pressure and subsequent drying. DE 228 247 also discloses the subsequent reaction of the morphinium methylsulfites obtained with metal halides or hydrohalic acids to give the corresponding morphinium halides.

JP 2001-322,970 describes the reaction of aliphatic trialkylamines with dimethyl sulfite in the presence of a polar solvent such as an alcohol or acetonitrile at from 40 to 100° C. to give the corresponding methyltrialkylammonium methylsulfites. The product was isolated by distilling off the solvent under reduced pressure. JP 2001-322,970 also discloses the subsequent reaction of the methyltrialkylammonium methylsulfites obtained with aqueous acid for the purpose of introducing the desired anion.

Compared to the other methylating agents listed above, dimethylsulfite has the great advantage of a sufficient methylation strength which makes mild reaction conditions possible and at the same time the relative ease with which most of the methylsulfite anion can be removed by heating after addition of the acid of the desired anion to form methanol and volatile sulfur dioxide. However, it was recognized according to the invention that the processes described in DE 228 247 and JP 2001-322,970 nevertheless leave a sulfur content of the order of ≧2% by weight in the isolated quaternary ammonium compound after reaction with the acid of the desired anion. However, this sulfur content interferes in various applications of the quaternary ammonium compound, in particular in its use in the electronics industry. The quaternary ammonium compounds prepared by the processes described in the prior art therefore have to be firstly subjected to costly purification before use, which represents a decisive disadvantage.

The anion exchange (second step of the synthesis) is usually carried out by reaction with
(i) the acid of the desired anion, in particular when the anion originally introduced in the methylation can be decomposed into products which can readily be separated off (e.g. methylcarbonate, methylsulfite); or when there is a significant difference in the solubility in a particular solvent or the crystallizability between the quaternary ammonium salt of the anion originally introduced in the methylation and the quaternary ammonium salt of the desired anion;
(ii) a compound which reacts with the anion originally introduced in the methylation or a replacement anion and thus forms the desired anion (e.g. replacement of Cl⁻ by F⁻ via addition of KF and phase transfer catalysis and reaction of the F⁻ with BF₃.OMe₂ to form [BF₄]⁻;
   or reaction of Br⁻ with Me—SO₂—OC₃H₇ to form Me—SO₃⁻);
(iii) a metal salt of the desired anion, particularly when the metal salt of the anion originally introduced in the methylation is very sparingly soluble (e.g. precipitation of silver chloride) or a significant enrichment of one phase of a hydrophilic/hydrophobic two-phase system with the quaternary ammonium salt having the desired anion (ion pair extraction) is possible;
(iv) silver hydroxide if the anion originally introduced in the methylation is chloride, bromide or iodide so as to introduce the hydroxide anion which can subsequently be neutralized with the acid of the desired anion;
(v) an insoluble (polymeric) anion exchanger.

An overview of various anion exchange variants is given in Houben-Weyl, Methoden der organischen Chemie, 4th edition, volume XI/2, Georg Thieme Verlag, Stuttgart 1958, pages 591 to 630 and Houben-Weyl, Methoden der organischen Chemie, expanded and supplementary volumes to 4th edition, volume E16a, part 2, Georg Thieme Verlag, Stuttgart 1990, pages 997 to 1017.

A disadvantage of anion exchange is the at least two-step synthesis which requires a high engineering outlay and makes only a reduced yield possible, not least because of the isolation of the intermediate. Furthermore, the handling of solids required in the abovementioned methods (iii) and (iv) is a disadvantage. In addition, the quaternary ammonium compound obtainable by anion exchange generally does not have the high purity required for use in the electronics industry, so that it usually has to be subjected to a costly further purification.

It was an object of the present invention to find a process for preparing quaternary ammonium compounds which does not have the disadvantages of the prior art, is simple to carry out, in which the alkylating agent to be used is nontoxic or only slightly toxic and which makes it possible for the desired anion to be introduced simply and flexibly. The quaternary ammonium compound having the desired anion should be able to be prepared in high purity and high yield without complicated purification steps and should also be suitable for use in the electronics industry.

We have accordingly found a process for preparing quaternary ammonium compounds by reacting the corresponding tertiary sp³-hybridized amine or sp²-hybridized imine with dimethyl sulfite, wherein the reaction is carried out
(i) in the presence of an inorganic or organic protic acid having a pKₐ of from 1.8 to 14, measured at 25° C. in aqueous solution; and
(ii) at a temperature of from 10 to 100° C.

The molar ratio of the inorganic or organic protic acid to the tertiary sp³-hybridized amine or sp²-hybridized imine in the process of the invention is generally from 0.9 to 1.5, preferably from 0.95 to 1.1, particularly preferably from 0.95 to 1.05 and very particularly preferably from 0.99 to 1.02.

In the process of the invention, preference is given to using an inorganic or organic protic acid whose partially or fully deprotonated anion is

fluoride; hexafluorophosphate; hexafluoroarsenate; hexafluoroantimonate; trifluoroarsenate; nitrite; nitrate; sulfate; hydrogensulfate; carbonate; hydrogencarbonate; phosphate; hydrogenphosphate; dihydrogenphosphate, vinylphosphonate, dicyanamide, bis(pentafluoroethyl)phosphinate, tris(pentafluoroethyl)trifluorophosphate, tris(heptafluoropropyl)trifluorophosphate, bis[oxalato(2−)]borate, bis[salicylato(2−))borate, bis[1,2-benzenediolato(2−)-O,O′]borate, tetracyanoborate, tetracarbonylcobaltate;

tetrasubstituted borate of the general formula (Va) [BR^{a}R^{b}R^{c}R^{d}]⁻, where R^{a} to R^{d} are each, independently of one another, fluorine or a carbon-comprising organic, saturated or unsaturated, acyclic or cyclic, aliphatic, aromatic or araliphatic radical which has from 1 to 30 carbon atoms and may comprise one or more heteroatoms and/or be substituted by one or more functional groups or halogen;

organic sulfonate of the general formula (Vb) [R^{e}—SO₃]⁻, where R^{e} is a carbon-comprising organic, saturated or unsaturated, acyclic or cyclic, aliphatic, aromatic or araliphatic radical which has from 1 to 30 carbon atoms and may comprise one or more heteroatoms and/or be substituted by one or more functional groups or halogen;

carboxylate of the general formula (Vc) [R^{f}—COO]⁻, where R^{f} is hydrogen or a carbon-comprising organic, saturated or unsaturated, acyclic or cyclic, aliphatic, aromatic or araliphatic radical which has from 1 to 30 carbon atoms and may comprise one or more heteroatoms and/or be substituted by one or more functional groups or halogen;

(fluoroalkyl)fluorophosphate of the general formula (Vd) [PFₓ(C_{y}F_{2y+1-z}H_{z})₆₋ₓ]⁻, where 1≦x≦6, 1≦y≦8 and 0≦z≦2y+1;

imide of the general formulae (Ve) [R^{g}—SO₂—N—SO₂—R^{h}]⁻, (Vf) [Rⁱ—SO₂—N—CO—R^{j}]⁻ or (IVg) [R^{k}—CO—N—CO—R^{l}]⁻, where R^{g} to R^{l} are each, independently of one another, hydrogen or a carbon-comprising organic, saturated or unsaturated, acyclic or cyclic, aliphatic, aromatic or araliphatic radical which has from 1 to 30 carbon atoms and may comprise one or more heteroatoms and/or be substituted by one or more functional groups or halogen;

methide of the general formula (Vh)

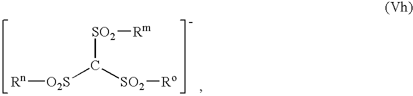

where R^{m} to R^{o} are, independently of one another, hydrogen or a carbon-comprising organic, saturated or unsaturated, acyclic or cyclic, aliphatic, aromatic or araliphatic radical which has from 1 to 30 carbon atoms and may comprise one or more heteroatoms and/or be substituted by one or more functional groups or halogen;

organic sulfate of the general formula (Vi) [R^{p}O—SO₃]⁻, where R^{p} is a carbon-comprising organic, saturated or unsaturated, acyclic or cyclic, aliphatic, aromatic or araliphatic radical which has from 1 to 30 carbon atoms and may comprise one or more heteroatoms and/or be substituted by one or more functional groups or halogen;

halometalate of the general formula (Vj) [M_{q}Halᵣ]^{s-}, where M is a metal and Hal is fluorine, chlorine, bromine or iodine, q and r are positive integers and indicate the stoichiometry of the complex and s is a positive integer and indicates the charge on the complex; or

sulfide, hydrogensulfide, hydrogenpolysulfide of the general formula (Vk) [HSᵥ]⁻, polysulfide of the general formula (Vm) [Sᵥ]²⁻, where v is a positive integer from 2 to 10, thiolate of the general formula (Vn) [R^{s}S]⁻, where R^{s} is a carbon-comprising organic, saturated or unsaturated, acyclic or cyclic, aliphatic, aromatic or araliphatic radical which has from 1 to 30 carbon atoms and may comprise one or more heteroatoms and/or be substituted by one or more functional groups or halogen.

Possible heteroatoms are in principle all heteroatoms which are able to formally replace a —CH₂— group, a —CH═ group, a C≡ group or a ═C═ group. If the carbon-comprising radical comprises heteroatoms, then preference is given to oxygen, nitrogen, sulfur, phosphorus and silicon. Preferred groups are, in particular, —O—, —S—, —SO—, —SO₂—, —NR—, —N═, —PR—, —PR₂ and —SiR₂—, where the radicals R are the remaining part of the carbon-comprising radical.

Possible functional groups are in principle all functional groups which can be bound to a carbon atom or a heteroatom. Examples of suitable groups are —OH (hydroxy), ═O (in particular as a carbonyl group), —NH₂ (amino), ═NH (imino), —COOH (carboxy), —CONH₂ (carboxamide) and —CN (cyano). Functional groups and heteroatoms can also be directly adjacent, so that combinations of a plurality of adjacent atoms, e.g. —O— (ether), —S— (thioether), —COO— (ester), —CONH— (secondary amide) or —CONR— (tertiary amide), are also encompassed.

As halogens, mention may be made of fluorine, chlorine, bromine and iodine.

Carbon-comprising organic, saturated or unsaturated, acyclic or cyclic, aliphatic, aromatic or araliphatic radicals having from 1 to 30 carbon atoms as the radicals R^{a} to R^{d} in the tetra-substituted borate (Va), the radical R^{e} in the organic sulfonate (Vb), the radical R^{f} in the carboxylate (Vc), the radicals R^{g} to R^{l} in the imides (Ve), (Vf) and (Vg), the radicals R^{m} to R^{o} in the methide (Vh), the radical R^{p} in the organic sulfate (Vi) and the radical R^{s} in the thiolate (Vn) are preferably, independently of one another,
C₁-C₃₀-alkyl and their aryl-, heteroaryl-, cycloalkyl-, halogen-, hydroxy-, amino-, carboxy-, formyl-, —O—, —CO—, —CO—O— or —CO—N←substituted components, for example methyl, ethyl, 1-propyl, 2-propyl, 1-butyl, 2-butyl, 2-methyl-1-propyl(isobutyl), 2-methyl-2-propyl(tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, heptyl, octyl, nonyl, decyl, undecyl, dodecyl, tridecyl, tetradecyl, pentadecyl, hexadecyl, heptadecyl, octadecyl, nonadecyl, icosyl, henicosyl, docosyl, tricosyl, tetracosyl, pentacosyl, hexacosyl, heptacosyl, octacosyl, nonacosyl, triacontyl, phenylmethyl(benzyl), diphenylmethyl, triphenylmethyl, 2-phenylethyl, 3-phenylpropyl, cyclopentylmethyl, 2-cyclopentylethyl, 3-cyclopentylpropyl, cyclohexylmethyl, 2-cyclohexylethyl, 3-cyclohexylpropyl, methoxy, ethoxy, formyl, acetyl or CₙF_{2(n-a)+(1-b)}H_{2a+b} where n≦30, 0≦a≦n and b=0 or 1 (for example CF₃, C₂F₅, CH₂CH₂—C(ₙ₋₂₎F₂₍ₙ₋₂₎₊₁, C₆F₁₃, C₈F₁₇, C₁₀F₂₁, C₁₂F₂₅);
C₃-C₁₂-cycloalkyl and their aryl-, heteroaryl-, cycloalkyl-, halogen-, hydroxy-, amino-, carboxy-, formyl-, —O—, —CO— or —CO—O-substituted components, for example cyclopentyl, 2-methyl-1-cyclopentyl, 3-methyl-1-cyclopentyl, cyclohexyl, 2-methyl-1-cyclohexyl, 3-methyl-1-cyclohexyl, 4-methyl-1-cyclohexyl or CₙF_{2(n-a)-(1-b)}H_{2a-b} where n≦30, 0≦a≦n and b=0 or 1;
C₂-C₃₀-alkenyl and their aryl-, heteroaryl-, cycloalkyl-, halogen-, hydroxy-, amino-, carboxy-, formyl-, —O—, —CO— or —CO—O-substituted components, for example 2-propenyl, 3-butenyl, cis-2-butenyl, trans-2-butenyl or CₙF_{2(n-a)-(1-b)}H_{2a-b} where n≦30, 0≦a≦n and b=0 or 1;
C₃-C₁₂-cycloalkenyl and their aryl-, heteroaryl-, cycloalkyl-, halogen-, hydroxy-, amino-, carboxy-, formyl-, —O—, —CO— or —CO—O-substituted components, for example 3-cyclopentenyl, 2-cyclohexenyl, 3-cyclohexenyl, 2,5-cyclohexadienyl or CₙF_{2(n-a)-3(1-b)}H_{2a-3b} where n≦30, 0≦a≦n and b=0 or 1; and
aryl or heteroaryl having from 2 to 30 carbon atoms and their alkyl-, aryl-, heteroaryl-, cycloalkyl-, halogen-, hydroxy-, amino-, carboxy-, formyl-, —O—, —CO— or —CO—O-substituted components, for example phenyl, 2-methylphenyl(2-tolyl), 3-methylphenyl(3-tolyl), 4-methylphenyl, 2-ethylphenyl, 3-ethylphenyl, 4-ethylphenyl, 2,3-dimethylphenyl, 2,4-dimethylphenyl, 2,5-dimethylphenyl, 2,6-dimethylphenyl, 3,4-dimethylphenyl, 3,5-dimethylphenyl, 4-phenylphenyl, 1-naphthyl, 2-naphthyl, 1-pyrrolyl, 2-pyrrolyl, 3-pyrrolyl, 2-pyridinyl, 3-pyridinyl, 4-pyridinyl or C₆F₍₅₋ₐ₎Hₐ where 0≦a≦5.

When the anion is a tetra-substituted borate (Va) [BR^{a}R^{b}R^{c}R^{d}]⁻, then all four radicals R^{a} to R^{d} in this are preferably identical and are preferably fluorine, trifluoromethyl, pentafluoroethyl, phenyl, 3,5-bis(trifluoromethyl)phenyl. Particularly preferred tetrasubstituted borates (Va) are tetrafluoroborate, tetraphenylborate and tetra[3,5-bis(trifluoromethyl)phenyl]borate.

When the anion is an organic sulfonate (Vb) [R^{e}—SO₃]⁻, then the radical R^{e} is preferably methyl, trifluoromethyl, pentafluoroethyl, p-tolyl or C₉F₁₉. Particularly preferred organic sulfonates (Vb) are trifluoromethanesulfonate(triflate), methanesulfonate, p-toluenesulfonate, nonadecafluorononanesulfonate(nonaflate), dimethylene glycol monomethyl ether sulfate and octylsulfate.

When the anion is a carboxylate (Vc) [R^{f}—COO]⁻, then the radical R^{f} is preferably hydrogen, trifluoromethyl, pentafluoroethyl, phenyl, hydroxyphenylmethyl, trichloromethyl, dichloromethyl, chloromethyl, trifluoromethyl, difluoromethyl, fluoromethyl, ethenyl(vinyl), 2-propenyl, —CH═CH—COO⁻, cis-8-heptadecenyl, —CH₂—C(OH)(COOH)—CH₂—COO⁻ or unbranched or branched C₁-C₁₈-alkyl, for example methyl, ethyl, 1-propyl, 2-propyl, 1-butyl, 2-butyl, 2-methyl-1-propyl(isobutyl), 2-methyl-2-propyl(tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, heptyl, octyl, nonyl, decyl, undecyl, dodecyl, heptadecyl. Particularly preferred carboxylates (Vc) are formate, acetate, propionate,. butyrate, valerate, benzoate, mandelate, trichloroacetate, dichloroacetate, chloroacetate, trifluoroacetate, difluoroacetate, fluoroacetate.

When the anion is a (fluoroalkyl)fluorophosphate (Vd) [PFₓ(C_{y}F_{2y+1-z}H_{z})_{6-x]}⁻, then z is preferably 0. Particular preference is given to (fluoroalkyl)fluorophosphates (Vd), in which z=0, x=3 and 1≦y≦4, specifically [PF₃(CF₃)₃]⁻, [PF₃(C₂F₅)₃]⁻, [PF₃(C₃F₇)₃]⁻ and [PF₃(C₄F₇)₃]⁻.

When the anion is an imide (Ve) [R^{g}—SO₂—N—SO₂—R^{h}]⁻, (Vf) [Rⁱ—SO₂—N—CO—R^{j}]⁻ or (Vg) [R^{k}—CO—N—CO—R^{j}]⁻, then the radicals R^{g} to R^{l} are each preferably, independently of one another, trifluoromethyl, pentafluoroethyl, phenyl, trichloromethyl, dichloromethyl, chloromethyl, trifluoromethyl, difluoromethyl, fluoromethyl or unbranched or branched C₁-C₁₂-alkyl, for example methyl, ethyl, 1-propyl, 2-propyl, 1-butyl, 2-butyl, 2-methyl-1-propyl(isobutyl), 2-methyl-2-propyl(tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, heptyl, octyl, nonyl, decyl, undecyl or dodecyl. Particularly preferred imides (Ve), (Vf) and (Vg) are [F₃C—SO₂—N—SO₂—CF₃]⁻ (bis(trifluoromethylsulfonyl)imide), [F₅C₂—SO₂—N—SO₂—C₂F₅]⁻ (bis(pentafluoroethylsulfonyl)imide), [F₃C—SO₂—N—CO—CF₃]⁻, [F₃C—CO—N—CO—CF₃]⁻ and those in which the radicals R^{g} to R^{l} are each, independently of one another, methyl, ethyl, propyl, butyl, phenyl, trichloromethyl, dichloromethyl, chloromethyl, trifluoromethyl, difluoromethyl or fluoromethyl.

When the anion is a methide (Vh)

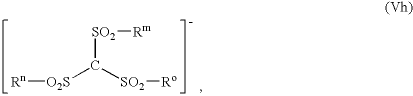

then the radicals R^{m} to R^{o} are each preferably, independently of one another, trifluoromethyl, pentafluoroethyl, phenyl, trichloromethyl, dichloromethyl, chloromethyl, trifluoromethyl, difluoromethyl, fluoromethyl or unbranched or branched C₁-C₁₂-alkyl, for example methyl, ethyl, 1-propyl, 2-propyl, 1-butyl, 2-butyl, 2-methyl-1-propyl(isobutyl), 2-methyl-2-propyl(tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, heptyl, octyl, nonyl, decyl, undecyl or dodecyl. Particularly preferred methides (Vh) are [(F₃C—SO₂)₃C]⁻ (tris(trifluoromethylsulfonyl)methide), [(F₅C₂—SO₂)₃C]⁻ (bis(pentafluoroethylsulfonyl)methide) and those in which the radicals R^{m} to R^{o} are each, independently of one another, methyl, ethyl, propyl, butyl, phenyl, trichloromethyl, dichloromethyl, chloromethyl, trifluoromethyl, difluoromethyl or fluoromethyl.

When the anion is an organic sulfate (Vi) [R^{p}O—SO₃]⁻, then the radical R^{p} is preferably a branched or unbranched C₁-C₃₀-alkyl radical. Particularly preferred organic sulfates (Vi) are methylsulfate, ethylsulfate, propylsulfate, butylsulfate, pentylsulfate, hexylsulfate, heptylsulfate or octylsulfate.

When the anion is a halometalate (Vj) [M_{q}Halᵣ]^{s-}, then M is preferably aluminum, zinc, iron, cobalt, antimony or tin. Hal is preferably chlorine or bromine and very particularly preferably chlorine. q is preferably 1, 2 or 3 and r and s are determined by the stoichiometry and charge on the metal ion.

When the anion is a thiolate (Vn) [R^{s}S]⁻, then the radical R^{s} is preferably a branched or unbranched C₁-C₃₀-alkyl radical. Particularly preferred thiolates (Vn) are methylsulfide, ethylsulfide, n-propylsulfide, n-butylsulfide, n-pentylsulfide, n-hexylsulfide, n-heptylsulfide, n-octylsulfide or n-dodecylsulfide.

The quaternary ammonium compound prepared in the process of the invention is very particularly preferably a quaternary ammonium salt in which the partially or fully deprotonated anion is tetrafluoroborate, hexafluorophosphate, trifluoromethanesulfonate, methanesulfonate, formate, acetate, mandelate, nitrate, nitrite, trifluoroacetate, sulfate, hydrogensulfate, methylsulfate, ethylsulfate, propylsulfate, butylsulfate, pentylsulfate, hexylsulfate, heptylsulfate, octylsulfate, phosphate, dihydrogenphosphate, hydrogenphosphate, propionate, tetrachloroaluminate, Al₂Cl₇⁻, chlorozincate, chloroferrate, bis(trifluoromethylsulfonyl)imide, bis(pentafluoroethylsulfonyl)imide, tris(trifluoromethylsulfonyl)methide, bis(pentafluoroethylsulfonyl)methide, p-toluenesulfonate, bis[salicylato(2−)]borate, tetracarbonylcobaltate, dimethylene glycol monomethyl ether sulfate, octylsulfate, oleate, stearate, acrylate, methacrylate, maleate, hydrogencitrate, vinylphosphonate, bis(pentafluoroethyl)phosphinate, bis[oxalato(2−)]borate, bis[1,2-benzenediolato(2−)-O,O′]borate, dicyanamide, tris(pentafluoroethyl)trifluorophosphate, tris(heptafluoropropyl)trifluorophosphate, tetracyanoborate or chlorocobaltate.

The pKₐ of the inorganic or organic protic acid to be used in the process of the invention is from 1.8 to 14, preferably from 1.8 to 10, particularly preferably from 2 to 10 and very particularly preferably from 3 to 10, measured at 25° C. in aqueous solution.

The molar ratio of dimethyl sulfite to the tertiary sp³-hybridized amine or sp²-hybridized imine in the process of the invention is generally from 0.9 to 1.5, preferably from 0.9 to 1.2, particularly preferably from 0.9 to 1.1 and very particularly preferably from 0.95 to 1.05 and in particular from 0.99 to 1.02.

The reaction between the tertiary sp³-hybridized amine or sp²-hybridized imine, the dimethyl sulfite and the inorganic or organic protic acid in the process of the invention is carried out at a temperature of from 10 to 100° C. and a pressure of from 0.05 to 2 MPa abs, preferably from 0.09 to 0.5 MPa abs, particularly preferably from 0.09 to 0.2 MPa abs and very particularly preferably from 0.095 to 0.12 MPa abs.

The time required for the reaction is dependent first and foremost on the chemical nature of the starting materials (reactivity of the tertiary sp³-hybridized amine or SP²-hybridized imine and the inorganic or organic protic acid) and the reaction temperature selected. It can be determined, for instance, by means of preliminary experiments in which, for example, the reaction kinetics are determined, the temperature curve of the exothermic reaction is measured and/or the concentrations of the starting materials and product are determined by analysis. In general, the time required is in the range from a few minutes to one day, generally of the order of from 0.5 to 24 hours, preferably of the order of from 0.5 to 10 hours.

As reaction apparatuses for the process of the invention, it is in principle possible to use all reaction apparatuses which are suitable for a reaction in the liquid phase. These are, in particular, reaction apparatuses which make appropriate mixing of the liquid starting materials possible, for example stirred vessels.

The type and order of the addition of the individual starting materials is not critical in the process of the invention. Thus, for example, it is possible to introduce the tertiary sp³-hybridized amine or sp²-hybridized imine, the dimethyl sulfite and the inorganic or organic protic acid into the reaction apparatus either in succession in any order or simultaneously. It is also possible to place one of the three starting materials in the reaction vessel initially and to add the other two starting materials dropwise over a particular period of time ranging from a few minutes to a number of hours.

In the process of the invention, it is also possible to carry out the reaction in the presence of a solvent. If a solvent is used, a solvent having a relatively low polarity is preferably chosen. Suitable solvents are, for example, aromatic hydrocarbons having from 6 to 10 carbon atoms, symmetrical or unsymmetrical dialkyl ethers having a total of from 5 to 10 carbon atoms, cycloalkanes having from 5 to 8 carbon atoms or C₅-C₁₀-alkanes. Specific examples are toluene, xylene, ethylbenzene, diethylbenzene, methyl tert-butyl ether, cyclohexane, hexane, heptane and octane. However, the reaction according to the invention is preferably carried out in the absence of solvents.

The reaction forms a sulfur dioxide and methanol, with most of the sulfur dioxide formed generally being given off during the reaction. Depending on the reaction temperature, the major part of the methanol formed is also given off during the reaction or remains in the reaction mixture. To free the reaction mixture of residual sulfur dioxide and methanol, it is generally advantageous to apply a vacuum to the reaction mixture after the reaction is complete and/or to heat it to a temperature above the boiling point of methanol and below the decomposition temperature of the quaternary ammonium compound. If no vacuum is available, the reaction mixture is preferably heated to a temperature of from 80 to 150° C. The conditions necessary for removal of residual sulfur dioxide and methanol can be determined in a simple manner by means of preliminary experiments in which the residual contents of sulfur dioxide and methanol and any possible decomposition products of the quaternary ammonium compound are advantageously analyzed or monitored.

Depending on the desired purity of the quaternary ammonium compound, it can be advantageous to subject the product obtained to a subsequent purification step. If the product is liquid at the working temperature, it can be shaken with a suitable solvent in which the quaternary ammonium compound is insoluble or only very slightly soluble. Suitable solvents for this purpose are generally solvents having a relatively low polarity, for example aromatic hydrocarbons having from 6 to 10 carbon atoms, symmetrical or unsymmetrical dialkyl ethers having a total of from 5 to 10 carbon atoms, cycloalkanes having from 5 to 8 carbon atoms or C₅-C₁₀-alkanes and also esters such as ethyl acetate. If the quaternary ammonium compound is solid at the working temperature, it can, for example, be washed with a suitable solvent in which the quaternary ammonium compound is insoluble or only very slightly soluble. Suitable solvents for this purpose are, for example, those which have been mentioned above. Furthermore, the solid quaternary ammonium compound can also be recrystallized from a suitable solvent. Suitable solvents for this purpose are solvents in which the quaternary ammonium compound dissolves, for example, alcohols, acetonitrile, tetrahydrofuran or nitrobenzene.

Depending on the further use of the optionally purified quaternary ammonium compound, it can be advantageous to dry it beforehand, for example under reduced pressure.

The process of the invention can be carried out batchwise, semicontinuously or continuously. When it is carried out batchwise, the starting materials are combined and the reaction is carried out at the desired temperature. After the reaction is complete, the reaction mixture is worked up as described. When it is carried out continuously, all three starting materials are slowly fed into the reaction apparatus for them to react at the desired temperature. The reaction mixture is taken off continuously in an amount corresponding to the amounts of starting materials fed in and is worked up as described. The work-up itself can likewise be carried out continuously. In the case of the semicontinuous variants, at least one starting material, preferably two or three starting materials, are slowly introduced at the desired temperature, with the reaction generally occurring in parallel with the addition. After the desired amount(s) has/have been added, the reaction mixture is generally left to react further for a particular time and is subsequently worked up as described.

In the process of the invention, the tertiary sp³-hybridized amine or tertiary sp²-hybridized imine used is preferably an amine, an imidazole, a pyridine or a guanidine.

In the process of the invention, preference is given to using an amine of the general formula (I)

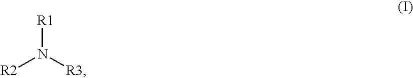

where

the radicals R¹ to R³ are each, independently of one another, a carbon-comprising organic, saturated or unsaturated, acyclic or cyclic, aliphatic, aromatic or araliphatic radical which has from 1 to 20 carbon atoms and is unsubstituted or interrupted or substituted by from 1 to 5 heteroatoms or functional groups, with the radical R¹ also being able to be hydrogen; or

the radical R¹ is as defined above and the radicals R² and R³ together form a divalent, carbon-comprising organic, saturated or unsaturated, acyclic or cyclic, aliphatic, aromatic or araliphatic radical which has from 1 to 30 carbon atoms and is unsubstituted or interrupted or substituted by from 1 to 5 heteroatoms or functional groups; or

the radicals R¹, R² and R³ together form a trivalent, carbon-comprising organic, saturated or unsaturated, acyclic or cyclic, aliphatic, aromatic or araliphatic radical which has from 1 to 40 carbon atoms and is unsubstituted or interrupted or substituted by from 1 to 5 heteroatoms or functional groups;

as tertiary sp³-hybridized amine.

In the process of the invention, preference is given to using an imidazole of the general formula (II)

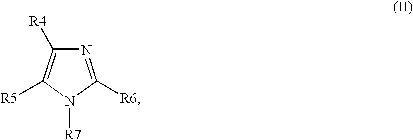

where

the radicals R⁴ to R⁷ are each, independently of one another, a carbon-comprising organic, saturated or unsaturated, acyclic or cyclic, aliphatic, aromatic or araliphatic radical which has from 1 to 20 carbon atoms and is unsubstituted or interrupted or substituted by from 1 to 5 heteroatoms or functional groups and the radicals R⁴ to R⁶ may also be, independently of one another, hydrogen, halogen or a functional group and the radical R⁷ may also be hydrogen; or

two adjacent radicals together form a divalent, carbon-comprising organic, saturated or unsaturated, acyclic or cyclic, aliphatic, aromatic or araliphatic radical which has from 1 to 30 carbon atoms and is unsubstituted or interrupted or substituted by from 1 to 5 heteroatoms or functional groups and the remaining radical is as defined above;

as tertiary sp²-hybridized imine.

In the process of the invention, preference is given to using a pyridine of the general formula (III)

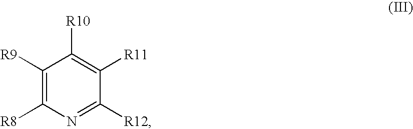

where

the radicals R⁸ to R¹² are each, independently of one another, hydrogen, halogen, a functional group or a carbon-comprising organic, saturated or unsaturated, acyclic or cyclic, aliphatic, aromatic or araliphatic radical which has from 1 to 20 carbon atoms and is unsubstituted or interrupted or substituted by from 1 to 5 heteroatoms or functional groups; or

in each case independently, two adjacent radicals together form a divalent, carbon-comprising organic, saturated or unsaturated, acyclic or cyclic, aliphatic, aromatic or araliphatic radical which has from 1 to 30 carbon atoms and is unsubstituted or interrupted or substituted by from 1 to 5 heteroatoms or functional groups and the remaining radicals/radical are/is as defined above;

as tertiary sp²-hybridized imine.

In the process of the invention, preference is given to using a guanidine of the general formula (IV)

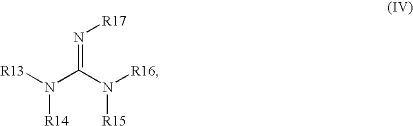

where

the radicals R¹³ to R¹⁷ are each, independently of one another, a carbon-comprising organic, saturated or unsaturated, acyclic or cyclic, aliphatic, aromatic or araliphatic radical which has from 1 to 20 carbon atoms and is unsubstituted or interrupted or substituted by from 1 to 5 heteroatoms or functional groups, with the radicals R¹³ and R¹⁵ also being able, independently of one another, to be hydrogen; or,

in each case independently, the radicals R¹³ and R¹⁴ and/or R¹⁵ and R¹⁶ together form a divalent, carbon-comprising organic, saturated or unsaturated, acyclic or cyclic, aliphatic, aromatic or araliphatic radical which has from 1 to 30 carbon atoms and is unsubstituted or interrupted or substituted by from 1 to 5 heteroatoms or functional groups and the remaining radicals/radical are/is as defined above; or

the radicals R¹⁴ and R¹⁵ together form a divalent, carbon-comprising organic, saturated or unsaturated, acyclic or cyclic, aliphatic, aromatic or araliphatic radical which has from 1 to 30 carbon atoms and is unsubstituted or interrupted or substituted by from 1 to 5 heteroatoms or functional groups and the remaining radicals are as defined above;

as tertiary sp²-hybridized imine.

Possible heteroatoms are in principle all heteroatoms in the definition of the radicals R¹ to R¹⁷ which are able to formally replace a —CH₂— group, a —CH═ group, a —C≡ group or a ═C═ group. If the carbon-comprising radical comprises heteroatoms, then preference is given to oxygen, nitrogen, sulfur, phosphorus and silicon. Preferred groups are, in particular, —O—, —S—, —SO—, —SO₂—, —NR—, —N═, —PR—, —PR₂ and —SiR₂—, where the radicals R are the remaining part of the carbon-comprising radical. In the case of R⁴ to R⁶ and R⁸ to R¹², the carbon-comprising radical can also be bound directly via the heteroatom to the imidazolium or pyridinium ring.

Possible functional groups are in principle all functional groups which can be bound to a carbon atom or a heteroatom. Examples of suitable groups are —OH (hydroxy), ═O (in particular as a carbonyl group), —NH₂ (amino), ═NH (imino), —COOH (carboxy), —CONH₂ (carboxamide), —SO₃H (sulfo) and —CN (cyano). Functional groups and heteroatoms can also be directly adjacent, so that combinations of a plurality of adjacent atoms, e.g. —O— (ether), —S— (thioether), —COO— (ester), —CONH— (secondary amide) or —CONR— (tertiary amide), are also encompassed, for example di(C₁-C₄-alkyl)amino, C₁-C₄-alkyloxycarbonyl or C₁-C₄-alkyloxy.

As halogen, mention may be made of fluorine, chlorine, bromine and iodine.

The process of the invention is preferably carried out using amines (I), imidazoles (II), pyridines (III) and guanidines (IV) in which the radicals R⁴ to R⁶ and R⁸ to R¹² are each, independently of one another,
hydrogen;
halogen; or
a functional group;

and the radicals R¹ to R¹⁷ are each, independently of one another,
C₁-C₁₈-alkyl which may optionally be substituted by functional groups, aryl, alkyl, aryloxy, alkyloxy, halogen, heteroatoms and/or heterocycles and/or be interrupted by one or more oxygen and/or sulfur atoms and/or one or more substituted or unsubstituted imino groups;
C₂-C₁₈-alkenyl which may optionally be substituted by functional groups, aryl, alkyl, aryloxy, alkyloxy, halogen, heteroatoms and/or heterocycles and/or be interrupted by one or more oxygen and/or sulfur atoms and/or one or more substituted or unsubstituted imino groups;
C₆-C₁₂-aryl which may optionally be substituted by functional groups, aryl, alkyl, aryloxy, alkyloxy, halogen, heteroatoms and/or heterocycles;
C₅-C₁₂-cycloalkyl which may optionally be substituted by functional groups, aryl, alkyl, aryloxy, alkyloxy, halogen, heteroatoms and/or heterocycles;
C₅-C₁₂-cycloalkenyl which may optionally be substituted by functional groups, aryl, alkyl, aryloxy, alkyloxy, halogen, heteroatoms and/or heterocycles; or
a five-membered to six-membered, oxygen-, nitrogen- and/or sulfur-containing heterocycle which may optionally be substituted by functional groups, aryl, alkyl, aryloxy, alkyloxy, halogen, heteroatoms and/or heterocycles; or

adjacent radicals R¹ and R², R² and R³, R¹ and R³, R⁴ and R⁵, R⁵ and R⁷, R⁷ and R⁶, R⁸ and R⁹, R⁹ and R¹⁰, R¹⁰ and R¹¹, R¹¹ and R¹², R¹³ and R¹⁴, R¹⁴ and R¹⁵, R¹⁵ and R¹⁶, R¹³ and R¹⁷ or R¹⁶ and R¹⁷ together form
an unsaturated, saturated or aromatic ring which may optionally be substituted by functional groups, aryl, alkyl, aryloxy, alkyloxy, halogen, heteroatoms and/or heterocycles and may optionally be interrupted by one or more oxygen and/or sulfur atoms and/or one or more substituted or unsubstituted imino groups.

C₁-C₁₈-Alkyl which may optionally be substituted by functional groups, aryl, alkyl, aryloxy, alkyloxy, halogen, heteroatoms and/or heterocycles is preferably methyl, ethyl, 1-propyl, 2-propyl, 1-butyl, 2-butyl, 2-methyl-1-propyl(isobutyl), 2-methyl-2-propyl(tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, heptyl, octyl, 2-ethylhexyl, 2,4,4-trimethylpentyl, 1,1,3,3-tetramethylbutyl, 1-nonyl, 1-decyl, 1-undecyl, 1-dodecyl, 1-tridecyl, 1-tetradecyl, 1-pentadecyl, 1-hexadecyl, 1-heptadecyl, 1-octadecyl, cyclopentylmethyl, 2-cyclopentylethyl, 3-cyclopentylpropyl, cyclohexylmethyl, 2-cyclohexylethyl, 3-cyclohexylpropyl, benzyl(phenylmethyl), diphenylmethyl(benzhydryl), triphenylmethyl, 1-phenylethyl, 2-phenylethyl, 3-phenylpropyl, α,α-dimethylbenzyl, p-tolylmethyl, 1-(p-butylphenyl)ethyl, p-chlorobenzyl, 2,4-dichlorobenzyl, p-methoxybenzyl, m-ethoxybenzyl, 2-cyanoethyl, 2-cyanopropyl, 2-methoxycarbonylethyl, 2-ethoxycarbonylethyl, 2-butoxycarbonylpropyl, 1,2-di(methoxycarbonyl)ethyl, methoxy, ethoxy, formyl, 1,3-dioxolan-2-yl, 1,3-dioxan-2-yl, 2-methyl-1,3-dioxolan-2-yl, 4-methyl-1,3-dioxolan-2-yl, 2-hydroxyethyl, 2-hydroxypropyl, 3-hydroxypropyl, 4-hydroxybutyl, 6-hydroxyhexyl, 2-aminoethyl, 2-aminopropyl, 3-aminopropyl, 4-aminobutyl, 6-aminohexyl, 2-methylaminoethyl, 2-methylaminopropyl, 3-methylaminopropyl, 4-methylaminobutyl, 6-methylaminohexyl, 2-dimethylaminoethyl, 2-dimethylaminopropyl, 3-dimethylaminopropyl, 4-dimethylaminobutyl, 6-dimethylaminohexyl, 2-hydroxy-2,2-dimethylethyl, 2-phenoxyethyl, 2-phenoxypropyl, 3-phenoxypropyl, 4-phenoxybutyl, 6-phenoxyhexyl, 2-methoxyethyl, 2-methoxypropyl, 3-methoxypropyl, 4-methoxybutyl, 6-methoxyhexyl, 2-ethoxyethyl, 2-ethoxypropyl, 3-ethoxypropyl, 4-ethoxybutyl, 6-ethoxyhexyl, acetyl, CₙF_{2(n-a)+(1-b)}H_{2a+b} where n is from 1 to 30, 0≦a≦n and b=0 or 1 (for example CF₃, C₂F₅, CH₂CH₂—C₍ₙ₋₂₎F₂₍ₙ₋₂₎₊₁, C₆F₁₃, C₈F₁₇, C₁₀F₂₁, C₁₂F₂₅), chloromethyl, 2-chloroethyl, trichloromethyl, 1,1-dimethyl-2-chloroethyl, methoxymethyl, 2-butoxyethyl, diethoxymethyl, diethoxyethyl, 2-isopropoxyethyl, 2-butoxypropyl, 2-octyloxyethyl, 2-methoxyisopropyl, 2-(methoxycarbonyl)ethyl, 2-(ethoxycarbonyl)ethyl, 2-(n-butoxycarbonyl)ethyl, butylthiomethyl, 2-dodecylthioethyl, 2-phenylthioethyl, 5-hydroxy-3-oxapentyl, 8-hydroxy-3,6-dioxaoctyl, 11-hydroxy-3,6,9-trioxaundecyl, 7-hydroxy-4-oxaheptyl, 11-hydroxy-4,8-dioxaundecyl, 15-hydroxy-4,8,12-trioxapentadecyl, 9-hydroxy-5-oxanonyl, 14-hydroxy-5,10-dioxatetradecyl, 5-methoxy-3-oxapentyl, 8-methoxy-3,6-dioxaoctyl, 11-methoxy-3,6,9-trioxaundecyl, 7-methoxy-4-oxaheptyl, 11-methoxy-4,8-dioxaundecyl, 15-methoxy-4,8,12-trioxapentadecyl, 9-methoxy-5-oxanonyl, 14-methoxy-5,10-dioxatetradecyl, 5-ethoxy-3-oxapentyl, 8-ethoxy-3,6-dioxaoctyl, 11-ethoxy-3,6,9-trioxaundecyl, 7-ethoxy-4-oxaheptyl, 11-ethoxy-4,8-dioxaundecyl, 15-ethoxy-4,8,12-trioxapentadecyl, 9-ethoxy-5-oxanonyl or 14-ethoxy-5,10-oxatetradecyl.

C₂-C₁₈-alkenyl which may optionally be substituted by functional groups, aryl, alkyl, aryloxy, alkyloxy, halogen, heteroatoms and/or heterocycles and/or be interrupted by one or more oxygen and/or sulfur atoms and/or one or more substituted or unsubstituted imino groups is preferably vinyl, 2-propenyl, 3-butenyl, cis-2-butenyl, trans-2-butenyl or CₙF_{2(n-a)-(1-b)}H_{2a-b} where n≦30, 0≦a≦n and b=0 or 1.

C₆-C₁₂-aryl which may optionally be substituted by functional groups, aryl, alkyl, aryloxy, alkyloxy, halogen, heteroatoms and/or heterocycles is preferably phenyl, tolyl, xylyl, α-naphthyl, β-naphthyl, 4-diphenylyl, chlorophenyl, dichlorophenyl, trichlorophenyl, difluorophenyl, methylphenyl, dimethylphenyl, trimethylphenyl, ethylphenyl, diethylphenyl, isopropylphenyl, tert-butylphenyl, dodecylphenyl, methoxyphenyl, dimethoxyphenyl, ethoxyphenyl, hexyloxyphenyl, methylnaphthyl, isopropylnaphthyl, chloronaphthyl, ethoxynaphthyl, 2,6-dimethylphenyl, 2,4,6-trimethylphenyl, 2,6-dimethoxyphenyl, 2,6-dichlorophenyl, 4-bromophenyl, 2-nitrophenyl, 4-nitrophenyl, 2,4-dinitrophenyl, 2,6-dinitrophenyl, 4-dimethylaminophenyl, 4-acetylphenyl, methoxyethylphenyl, ethoxymethylphenyl, methylthiophenyl, isopropylthiophenyl or tert-butylthiophenyl or C₆F₍₅₋ₐ₎Hₐ where 0≦a≦5.

C₅-C₁₂-cycloalkyl which may optionally be substituted by functional groups, aryl, alkyl, aryloxy, alkyloxy, halogen, heteroatoms and/or heterocycles is preferably cyclopentyl, cyclohexyl, cyclooctyl, cyclododecyl, methylcyclopentyl, dimethylcyclopentyl, methylcyclohexyl, dimethylcyclohexyl, diethylcyclohexyl, butylcyclohexyl, methoxycyclohexyl, dimethoxycyclohexyl, diethoxycyclohexyl, butylthiocyclohexyl, chlorocyclohexyl, dichlorocyclohexyl, dichlorocyclopentyl, CₙF_{2(n-a)-(1-b)}H_{2a-b} where n≦30, 0≦a≦n and b=0 or 1 or a saturated or unsaturated bicyclic system such as norbornyl or norbornenyl.

C₅-C₁₂-cycloalkenyl which may optionally be substituted by functional groups, aryl, alkyl, aryloxy, alkyloxy, halogen, heteroatoms and/or heterocycles is preferably 3-cyclopentenyl, 2-cyclohexenyl, 3-cyclohexenyl, 2,5-cyclohexadienyl or CₙF_{2(n-a)-3(1-b)}H_{2a-3b} where n≦30, 0≦a≦n and b=0 or 1.

A five-membered to six-membered, oxygen-, nitrogen- and/or sulfur-containing heterocycle which may optionally be substituted by functional groups, aryl, alkyl, aryloxy, alkyloxy, halogen, heteroatoms and/or heterocycles is preferably furyl, thiophenyl, pyrryl, pyridyl, indolyl, benzoxazolyl, dioxolyl, dioxyl, benzimidazolyl, benzthioazolyl, dimethylpyridyl, methylquinolyl, dimethylpyrryl, methoxyfuryl, dimethoxypyridyl or difluoropyridyl.

If the adjacent radicals R¹ and R², R² and R³, R¹ and R³, R⁴ and R⁵, R⁵ and R⁷, R⁷ and R⁶, R⁸ and R⁹, R⁹ and R¹⁰, R¹⁰ and R¹¹, R¹¹ and R¹², R¹³ and R¹⁴, R¹⁴ and R¹⁵, R¹⁵ and R¹⁶, R¹³ and R¹⁷ or R¹⁶ and R¹⁷ together form an unsaturated, saturated or aromatic ring which may optionally be substituted by functional groups, aryl, alkyl, aryloxy, alkyloxy, halogen, heteroatoms and/or heterocycles and may optionally be interrupted by one or more oxygen and/or sulfur atoms and/or one or more substituted or unsubstituted imino groups, the two radicals together are preferably 1,3-propylene, 1,4-butylene, 1,5-pentylene, 2-oxa-1,3-propylene, 1-oxa-1,3-propylene, 2-oxa-1,3-propylene, 1-oxa-1,3-propenylene, 3-oxa-1,5-pentylene, 1-aza-1,3-propenylene, 1-C₁-C₄-alkyl-1-aza-1,3-propenylene, 1,4-buta-1,3-dienylene, 1-aza-1,4-buta-1,3-dienylene or 2-aza-1,4-buta-1,3-dienylene.

If the abovementioned radicals comprise oxygen and/or sulfur atoms and/or substituted or unsubstituted imino groups, the number of oxygen and/or sulfur atoms and/or imino groups is not subject to any restrictions. In general, there will be no more than 5 in the radical, preferably no more than 4 and very particularly preferably no more than 3.

If the abovementioned radicals comprise heteroatoms, there is generally at least one carbon atom, preferably at least two carbon atoms, between any two heteroatoms.

The radicals R¹ to R³, R⁷ and R¹³ to R¹⁷ are particularly preferably, independently of one another, unbranched or branched C₁-C₁₂-alkyl, for example methyl, ethyl, 1-propyl, 2-propyl, 1-butyl, 2-butyl, 2-methyl-1-propyl(isobutyl), 2-methyl-2-propyl(tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, 1-heptyl, 1-octyl, 1-nonyl, 1-decyl, 1-undecyl, 1-dodecyl, 1-tetradecyl, 1-hexadecyl, 1-octadecyl, 2-hydroxyethyl, benzyl, 3-phenylpropyl, vinyl, 2-cyanoethyl, 2-(methoxycarbonyl)ethyl, 2-(ethoxycarbonyl)ethyl, 2-(n-butoxycarbonyl)ethyl, dimethylamino, diethylamino, trifluoromethyl, difluoromethyl, fluoromethyl, pentafluoroethyl, heptafluoropropyl, heptafluoroisopropyl, nonafluorobutyl, nonafluoroisobutyl, undecylfluoropentyl, undecylfluoroisopentyl, 6-hydroxyhexyl or propylsulfonic acid. In addition, particular preference is also given to the radical R⁷ being a sulfo group or an unbranched or branched sulfo-C₁-C₁₂-alkyl radical.

The radicals R⁴ to R⁶ and R⁸ to R¹² are particularly preferably, independently of one another, hydrogen or unbranched or branched C₁-C₁₂-alkyl, for example methyl, ethyl, 1-propyl, 2-propyl, 1-butyl, 2-butyl, 2-methyl-1-propyl(isobutyl), 2-methyl-2-propyl(tert-butyl), 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,2-dimethyl-1-butyl, 2,3-dimethyl-1-butyl, 3,3-dimethyl-1-butyl, 2-ethyl-1-butyl, 2,3-dimethyl-2-butyl, 3,3-dimethyl-2-butyl, heptyl, octyl, nonyl, decyl, undecyl, dodecyl, 2-hydroxyethyl, 2-cyanoethyl, 2-(methoxycarbonyl)ethyl, 2-(ethoxycarbonyl)ethyl, 2-(n-butoxycarbonyl)ethyl, dimethylamino, diethylamino, chlorine, trifluoromethyl, difluoromethyl, fluoromethyl, pentafluoroethyl, heptafluoropropyl, heptafluoroisopropyl, nonafluorobutyl, nonafluoroisobutyl, undecylfluoropentyl, undecylfluoroisopentyl or 6-hydroxyhexyl.

Very particular preference is given to using trimethylamine, dimethylethylamine, dimethyl-n-propylamine, diethylmethylamine, triethylamine, tri-n-propylamine, di-n-propylmethylamine, tri-n-butylamine, di-n-butylmethylamine, tri-n-pentylamine, N-methylpiperidine, N,N-dimethylaniline and N-methylmorpholine as amine (I) in the process of the invention.

Very particular preference is given to using N-methylimidazole, N-ethylimidazole, N-(1-propyl)imidazole, N-(1-butyl)imidazole, N-(1-hexyl)imidazole, N-(1-octyl)imidazole, N-(1-decyl)imidazole, N-(1-dodecyl)imidazole and N-(1-pentadecyl)imidazole as imidazole (II) in the process of the invention.

Very particular preference is given to using pyridine, 2-methylpyridine, 3-methylpyridine, 4-methylpyridine, 2,4-dimethylpyridine, 2,6-dimethylpyridine, 2-ethylpyridine and 2,6-diethylpyridine as pyridine (III) in the process of the invention.

Very particular preference is given to using N,N,N′,N′,N″-pentamethylguanidine as guanidine (IV) in the process of the invention.

If amines are used in the process of the invention, the reaction between these, the dimethyl sulfite and the inorganic or organic protic acid is preferably carried out at a temperature of from 10 to 80° C., particularly preferably from 10 to 60° C. and very particularly preferably from 10 to 40° C.

If imidazoles, pyridines or guanidines are used in the process of the invention, the reaction between these, the dimethyl sulfite and the inorganic or organic protic acid is preferably carried out at a temperature of from 20 to 100° C., particularly preferably from 30 to 90° C. and very particularly preferably from 50 to 80° C.

In a general embodiment, one of the three starting materials is placed in a reaction vessel and the other two starting materials are fed in simultaneously with mixing at the desired temperature and the desired pressure over a period of a few minutes to a number of hours. After the addition is complete, the reaction mixture is generally left for a further period ranging from some minutes to a number of hours while continuing to stir. During this time, it is advantageous to apply a vacuum and/or increase the temperature to up to 150° C. so as to separate off the residual sulfur dioxide and methanol. The quaternary ammonium compound obtained is preferably washed with a suitable solvent and subsequently dried under reduced pressure.

The process of the invention makes it possible to prepare quaternary ammonium compounds having a flexibly selectable anion in high purity without complicated purification steps, is simple to carry out and, due to the use of dimethyl sulfite as methylating agent, requires no toxic substances. Despite the use of dimethyl sulfite, rearrangement of the methylsulfite formed to methanesulfonate is virtually completely avoided or at least significantly suppressed in the process of the invention, which is decisive in making possible the high purity of the quaternary ammonium compounds in respect of the possible by-product anion methanesulfonate, too. In contrast thereto, the quaternary ammonium compounds prepared according to the prior art contain significant amounts of the methanesulfonate anion which has been formed by rearrangement of the methylsulfite anion and can no longer be decomposed into volatile components.

The quaternary ammonium compounds which can be prepared by the process of the invention can therefore be used without problems in the electronics industry.

### EXAMPLES

### Example 1 (According to the Invention)

21.11 g (0.192 mol) of dimethyl sulfite were placed in a reaction vessel at room temperature and 23.8 g (0.192 mol) of N-butylimidazole and 12 g (0.2 mol) of acetic acid were added dropwise over a period of 25 minutes while stirring. The reaction mixture was stirred at 60° C. for 15 hours and subsequently heated at 120° C. for 4 hours, with residual sulfur dioxide and methanol being distilled off. The reaction product obtained was dried at 100° C. under a reduced pressure of 0.3 kPa (3 mbar). The yield was 35.5 g, corresponding to 93% of the theoretic total yield.

The liquid product obtained was analyzed by NMR spectroscopy and identified as N,N′-butylmethylimidazolium acetate:

[1H-NMR, 400 Mhz], D₂O.: 0.9 ppm (t-3H); 1.3 ppm (m-2H); 1.8 ppm (m-2H); 1.9 ppm (s-3H CH₃COO⁻); 2.8 ppm (s-3H—CH₃SO₃⁻); 3.4 ppm (s-3H); 3.8 ppm (s-3H); 4.2 ppm (t-2H); 7.4 ppm (d-2H); 8.7 ppm (s-1H)

In a quantitative evaluation of the NMR spectrum, the ratio of the signals 2.8 ppm (3H-methanesulfonate): 3.8 ppm (3H-methyl group on the imidazolium nitrogen) indicated that the proportion of methanesulfonate formed was below the detection limit. This is 3 mol %. The purity of the N,N′-butylmethylimidazolium acetate was thus >97%.

### Example 2 (Comparative Example)

62 g (0.5 mol) of N-butylimidazole was mixed with 55 g (0.5 mol) of dimethyl sulfite in a 250 ml four-neck flask at room temperature and the mixture was heated to 80° C. The reaction mixture was stirred for 5 hours and then cooled. The cooled reaction mixture was shaken twice with ethyl acetate and subsequently dried under reduced pressure. The yield obtained was 108.6 g, corresponding to 92.8% of the theoretical total yield (N,N′-butylmethylimidazolium methylsulfite and N,N′-butylmethylimidazolium methanesulfonate).

13 g (0.21 mol) of acetic acid were then added to 50.3 g (0.21 mol) of this product mixture and the reaction mixture was heated to 110° C. Gentle reflux was observed. The volatile components (methanol and sulfur dioxide) were removed under reduced pressure. The reaction product was subsequently dried at 140° C. under reduced pressure. The yield obtained was 37.6 g, corresponding to 90% of the theoretical yield based on the N,N′-butylmethylimidazolium methylsulfite and N,N′-butylmethylimidazolium methanesulfonate used.

The calculated total yield based on the N-butylimidazole used was 92.8% 90%=83.5%.

The liquid product obtained was analyzed by NMR spectroscopy and identified as a mixture of N,N′-butylmethylimidazolium acetate and N,N′-butylmethylimidazolium methanesulfonate:

[1H-NMR, 400Mhz], D₂O.: 0.9 ppm (t-3H); 1.3 ppm (m-2H); 1.8 ppm (m-2H); 1.9 ppm (s-3H CH₃COO⁻); 2.8 ppm (s-3H—CH₃SO₃⁻); 3.4 ppm (s-3H); 3.8 ppm (s-3H); 4.2 ppm (t-2H); 7.4 ppm (d-2H); 8.7 ppm (s-1H)

In a quantitative evaluation of the NMR spectrum, the ratio of signals 2.8 ppm (3H-methanesulfonate): 3.8 ppm (3H-methyl group on the imidazolium nitrogen) indicated that the proportion of methanesulfonate formed was 21 mol %. The purity of the N,N′-butylmethylimidazolium acetate was thus only 79%.

Comparison of examples 1 and 2 shows that N,N′-butylmethylimidazolium acetate can be obtained in a purity of >97% and a total yield of 93% by means of the process of the invention, while the two-step synthesis of example 2 made it possible to achieve only a purity of 79% and a total yield of 83.5%.

### Example 3 (Comparative Example Using Acetonitrile as Solvent)

Example 3 was carried out using a procedure which was substantially analogous to example 1 of JP 2001-322,970.

20.0 g (0.198 mol) of triethylamine, 21.8 g (0.198 mol) of dimethyl sulfite and 40 ml of acetonitrile were combined and refluxed for 2 hours under atmospheric pressure while stirring. The acetonitrile was subsequently distilled off under reduced pressure and the liquid triethylmethylammonium salt was obtained. This was dissolved in 100 ml of water and admixed with 38.0 g of 50% strength aqueous tetrafluoroboric acid, corresponding to 0.198 mol of HBF₄. This solution was heated to 70° C., with the sulfur dioxide formed being given off. After evolution of sulfur dioxide had ceased, water and methanol were distilled off under reduced pressure. The theoretical total yield was 92% (triethylmethylammonium methylsulfite and triethylmethylammonium methanesulfonate).

Compared to example 1 of JP 2001-322,970, in which a yield of 96% is reported, the yield in the repetition of the experiment was only 92%.

The liquid product obtained was analyzed by NMR spectroscopy and identified as a mixture of triethylmethylammonium methylsulfite and triethylmethylammonium methanesulfonate:

[1H-NMR, 400 Mhz], D₂O.: 1.3 ppm (t-9H); 2.8 ppm (s-3H-methanesulfonate); 2.9 ppm (s-3H); 3.3 ppm (q-6H)

In addition, the NMR spectrum was evaluated quantitatively and the proportion of methanesulfonate formed was calculated as 6.1 mol % from the ratio of signals 2.8 ppm (3H-methanesulfonate): 2.9 ppm (3H-methyl group on the ammonium nitrogen). The purity of the triethylmethylammonium methylsulfite was thus only 93.9%.

### Example 4 (Comparative Example Using Acetonitrile as Solvent)

Example 4 was carried out using a procedure which was substantially analogous to example 1 of JP 2001-322,970, but pyridine was used in place of triethylamine.

15.66 g (0.198 mol) of pyridine, 21.8 g (0.198 mol) of dimethyl sulfite and 40 ml of acetonitrile were combined and refluxed for 2 hours under atmospheric pressure while stirring. The acetonitrile was subsequently distilled off under reduced pressure and the liquid methylpyridinium salt was obtained. This was dissolved in 100 ml of water and admixed with 38.0 g of 50% strength aqueous tetrafluoroboric acid, corresponding to 0.198 mol of HBF₄. This solution was heated to 70° C., with the sulfur dioxide formed being given off. After evolution of sulfur dioxide had ceased, water and methanol were distilled off under reduced pressure. The theoretical total yield was 86.8% (methylpyridinium methylsulfite and methylpyridinium methanesulfonate).

The liquid product obtained was analyzed by NMR spectroscopy and identified as a mixture of methylpyridinium methylsulfite and methylpyridinium methanesulfonate:

[1H-NMR, 400 Mhz], D₂O.: 2.8 ppm (s-3H-methanesulfonate); 4.4 ppm (s-3H); 4.45 ppm (s-3H-secondary components); 8.0 ppm (m, 2H); 8.5 ppm (m-1H); 8.8 ppm (m-2H)

In addition, the NMR spectrum was evaluated quantitatively and the proportion of methanesulfonate formed was calculated as 10.5 mol % from the ratio of signals 2.8 ppm (3H-methanesulfonate): 4.4 ppm (3H-methyl group on the pyridinium nitrogen). The purity of the pyridinium methylsulfite was thus only 89.5%.

1-10. (canceled)

11. A process for preparing quaternary ammonium compounds comprising the step of reacting a tertiary sp³-hybridized amine or a sp²-hybridized imine with dimethyl sulfite, wherein the reaction is carried out
(i) in the presence of an inorganic or organic protic acid having a pKₐ of from 1.8 to 14 measured at 25° C. in aqueous solution; and
(ii) at a temperature of from 10° C. to 100° C.

12. The process according to claim 11, wherein the molar ratio of said inorganic or organic protic acid to said tertiary sp³-hybridized amine or sp²-hybridized imine is from 0.9 to 1.5.

13. The process according to claim 11, wherein the partially or fully deprotonated anion of said inorganic or organic protic acid is fluoride; hexafluorophosphate; hexafluoroarsenate; hexafluoroantimonate; trifluoroarsenate; nitrite; nitrate; sulfate; hydrogensulfate; carbonate; hydrogencarbonate; phosphate; hydrogenphosphate; dihydrogenphosphate; vinylphosphonate; dicyanamide; bis(pentafluoroethyl)phosphinate; tris(pentafluoroethyl)trifluorophosphate; tris(heptafluoropropyl)trifluorophosphate; bis[oxalato(2−)]borate; bis[salicylato(2−)]borate; bis[1,2-benzenediolato(2−)-O,O′]borate; tetracyanoborate; tetracarbonylcobaltate;
tetrasubstituted borate of the general formula (Va)
[BR^{a}R^{b}R^{c}R^{d}]⁻ (Va)
wherein R^{a}, R^{b}, R^{c}, and R^{d} are each, independently one another, fluorine or a saturated or
unsaturated, acyclic or cyclic, aliphatic, aromatic or araliphatic radical having up to 30 carbon atoms, wherein said radical is optionally substituted with one or more functional groups or halogen and wherein one or more carbon atoms of said radical is optionally replaced with heteroatoms;
organic sulfonate of the general formula (Vb)
[R^{e}—SO₃]⁻ (Vb)
wherein R^{e} is a saturated or unsaturated, acyclic or cyclic, aliphatic, aromatic or araliphatic radical having up to 30 carbon atoms, wherein said radical is optionally substituted with one or more functional groups or halogen and wherein one or more carbon atoms of said radical is optionally replaced with heteroatoms;
carboxylate of the general formula (Vc)
[R^{f}—COO]⁻ (Vc)
wherein R^{f} is hydrogen or a saturated or unsaturated, acyclic or cyclic, aliphatic, aromatic or araliphatic radical having up to 30 carbon atoms, wherein said radical is optionally substituted with one or more functional groups or halogen and wherein one or more carbon atoms of said radical is optionally replaced with heteroatoms;
(fluoroalkyl)fluorophosphate of the general formula (Vd)
[PFₓ(C_{y}F_{2y+1-z}H_{z})₆₋ₓ]⁻ (Vd)
wherein 1≦x≦6, 1≦y≦8 and0≦z≦2y+1;
imide of the general formulae (Ve), (Vf), and (Vg)
[R^{g}—SO₂—N—SO₂—R^{h}]⁻ (Ve)
[Rⁱ—SO₂—N—CO—R^{j}]⁻ (Vf)
[R^{k}—CO—N—CO—R^{l}]⁻ (Vg)
wherein R^{g}, R^{h}, Rⁱ, R^{j}, R^{k}, and R^{l} are each, independently of one another, hydrogen or a saturated or unsaturated, acyclic or cyclic, aliphatic, aromatic or araliphatic radical having up to 30 carbon atoms, wherein said radical is optionally substituted with one or more functional groups or halogen and wherein one or more carbon atoms of said radical is optionally replaced with heteroatoms;
methide of the general formula (Vh) 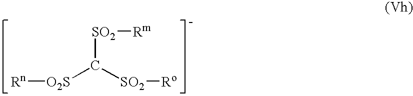
wherein R^{m}, Rⁿ, and R^{o} are each, independently of one another, hydrogen or a saturated or unsaturated, acyclic or cyclic, aliphatic, aromatic or araliphatic radical having up to 30 carbon atoms, wherein said radical is optionally substituted with one or more functional groups or halogen and wherein one or more carbon atoms of said radical is optionally replaced with heteroatoms;
organic sulfate of the general formula (Vi)
[R^{p}O—S₃]⁻ (Vi)
wherein R^{p} is a saturated or unsaturated, acyclic or cyclic, aliphatic, aromatic or araliphatic radical which has from 1 to 30 carbon atoms, wherein said radical is optionally substituted with one or more functional groups or halogen and wherein one or more carbon atoms of said radical is optionally replaced with heteroatoms;
halometalate of the general formula (Vj)
[M_{q}Halᵣ]^{s-} (Vj)
wherein
M is a metal
Hal is fluorine, chlorine, bromine or iodine, and
q, r, and s are positive integers; or
sulfide, hydrogensulfide, hydrogenpolysulfide of the general formula (Vk)
[HSᵥ]⁻ (Vk)
wherein v is a positive integer from 2 to 10;
polysulfide of the general formula (Vm)
[Sᵥ]²⁻ (Vm)
wherein v is a positive integer from 2 to 10;
thiolate of the general formula (Vn)
[R^{s}S]⁻ (Vn)
wherein R^{s} is a saturated or unsaturated, acyclic or cyclic, aliphatic, aromatic or araliphatic radical having up to 30 carbon atoms, wherein said radical is optionally substituted with one or more functional groups or halogen and wherein one or more carbon atoms of said radical is optionally replaced with heteroatoms.

14. The process according to claim 13, wherein the partially or fully deprotonated anion of said inorganic or organic protic acid is tetrafluoroborate, hexafluorophosphate, trifluoromethanesulfonate, methanesulfonate, formate, acetate, mandelate, nitrate, nitrite, trifluoroacetate, sulfate, hydrogensulfate, methylsulfate, ethylsulfate, propylsulfate, butylsulfate, pentylsulfate, hexylsulfate, heptylsulfate, octylsulfate, phosphate, dihydrogenphosphate, hydrogenphosphate, propionate, tetrachloroaluminate, Al₂Cl₇⁻, chlorozincate, chloroferrate, bis(trifluoromethylsulfonyl)imide, bis(pentafluoroethylsulfonyl)imide, tris(trifluoromethylsulfonyl)methide, bis(pentafluoroethylsulfonyl)methide, p-toluenesulfonate, bis[salicylato(2−)]borate, tetracarbonylcobaltate, dimethylene glycol monomethyl ether sulfate, octylsulfate, oleate, stearate, acrylate, methacrylate, maleate, hydrogencitrate, vinylphosphonate, bis(pentafluoroethyl)phosphinate, bis[oxalato(2−)]borate, bis[1,2-benzenediolato(2−)-O,O′]borate, dicyanamide, tris(pentafluoroethyl)trifluorophosphate, tris(heptafluoropropyl)trifluorophosphate, tetracyanoborate or chlorocobaltate.

15. The process according to claim 11, wherein the molar ratio of said dimethyl sulfite to said tertiary sp³-hybridized amine or sp²-hybridized imine is from 0.9 to 1.5.

16. The process according to claim 11, wherein said tertiary sp³-hybridized amine is an amine of the general formula (I) 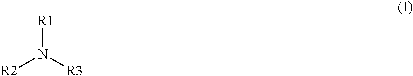
wherein
R¹, R², and R³ are each, independently of one another, a saturated or unsaturated, acyclic or cyclic, aliphatic, aromatic or araliphatic radical having up to 20 carbon atoms, wherein said radical is optionally substituted with up to 5 functional groups and wherein up to 5 of the carbon atoms of said radical is optionally replaced with heteroatoms; and wherein R¹ is optionally hydrogen; or
R¹ is hydrogen or a saturated or unsaturated, acyclic or cyclic, aliphatic, aromatic or araliphatic radical having up to 20 carbon atoms, wherein said radical is optionally substituted with up to 5 functional groups and wherein up to 5 of the carbon atoms of said radical is optionally replaced with heteroatoms; and R² and R³ together form a divalent, saturated or unsaturated, acyclic or cyclic, aliphatic, aromatic or araliphatic radical having up to 30 carbon atoms, wherein said divalent radical is optionally substituted with up to 5 functional groups and wherein up to 5 of the carbon atoms of said divalent radical is optionally replaced with heteroatoms; or
R¹, R², and R³ together form a trivalent, saturated or unsaturated, acyclic or cyclic, aliphatic, aromatic or araliphatic radical having up to 40 carbon atoms, wherein said trivalent radical is optionally substituted with up to 5 functional groups and wherein up to 5 of the carbon atoms of said trivalent radical is optionally replaced with heteroatoms.

17. The process according to claim 11, wherein said tertiary sp²-hybridized imine is an imidazole of the general formula (II) 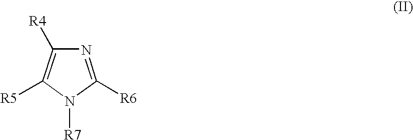
wherein
R⁴, R⁵, R⁶, and R⁷ are each, independently of one another, hydrogen, a sulfo group, or a saturated or unsaturated, acyclic or cyclic, aliphatic, aromatic or araliphatic radical having up to 20 carbon atoms, wherein said radical is optionally substituted with up to 5 functional groups and wherein up to 5 of the carbon atoms of said radical is optionally replaced with heteroatoms; and wherein R⁴, R⁵, and R⁶ are optionally, independently of one another, halogen; or
R⁴ and R⁵ or R⁵ and R⁶ or R⁶ and R⁷ together form a divalent, saturated or unsaturated, acyclic or cyclic, aliphatic, aromatic or araliphatic radical having up to 30 carbon atoms, wherein said divalent radical is optionally substituted with up to 5 functional groups and wherein up to 5 of the carbon atoms of said divalent radical is optionally replaced with heteroatoms; and the remaining substituents are each, independently of one another, hydrogen, a sulfo group, or a saturated or unsaturated, acyclic or cyclic, aliphatic, aromatic or araliphatic radical having up to 20 carbon atoms, wherein said radical is optionally substituted with up to 5 functional groups and wherein up to 5 of the carbon atoms of said radical is optionally replaced with heteroatoms, and optionally halogen when R⁴, R⁵, or R⁶ are remaining substituents.

18. The process according to claim 11, wherein said tertiary sp²-hybridized imine is a pyridine of the general formula (III) 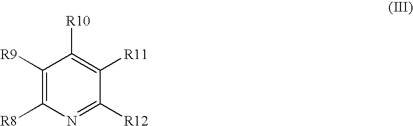
wherein
R⁸, R⁹, R¹⁰, R¹¹, and R¹² are each, independently of one another, hydrogen, halogen, a functional group or a saturated or unsaturated, acyclic or cyclic, aliphatic, aromatic or araliphatic radical having up to 20 carbon atoms, wherein said radical is optionally substituted with up to 5 functional groups and wherein up to 5 of the carbon atoms of said radical is optionally replaced with heteroatoms; or
R⁸ and R⁹ or R⁹ and R¹⁰ or R¹⁰ and R¹¹ or R¹¹ and R¹² together form a divalent, saturated or unsaturated, acyclic or cyclic, aliphatic, aromatic or araliphatic radical having up to 30 carbon atoms, wherein said divalent radical is optionally substituted with up to 5 functional groups and wherein up to 5 of the carbon atoms of said divalent radical is optionally replaced with heteroatoms; and the remaining substituents are each, independently of one another, hydrogen, halogen, a functional group or a saturated or unsaturated, acyclic or cyclic, aliphatic, aromatic or araliphatic radical having up to 20 carbon atoms, wherein said radical is optionally substituted with up to 5 functional groups and wherein up to 5 of the carbon atoms of said radical is optionally replaced with heteroatoms.

19. The process according to claim 11, wherein said tertiary sp²-hybridized imine is a guanidine of the general formula (IV) 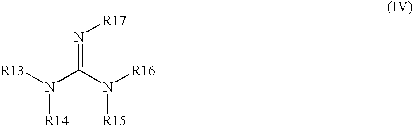
wherein
R¹³, R¹⁴, R¹⁵, R¹⁶, and R¹⁷ are each, independently of one another, a saturated or unsaturated, acyclic or cyclic, aliphatic, aromatic or araliphatic radical having up to 20 carbon atoms, wherein said radical is optionally substituted with up to 5 functional groups and wherein up to 5 of the carbon atoms of said radical is optionally replaced with heteroatoms; and wherein R¹³ and R¹⁵ are optionally, independently of one another, hydrogen; or
R¹³ and R¹⁴ and/or R¹⁵ and R¹⁶ together form, independently in each case, a divalent, saturated or unsaturated, acyclic or cyclic, aliphatic, aromatic or araliphatic radical having up to 30 carbon atoms, wherein said divalent radical is optionally substituted with up to 5 functional groups and wherein up to 5 of the carbon atoms of said divalent radical is optionally replaced with heteroatoms; and the remaining substituent or substituents are each, independently of one another, a saturated or unsaturated, acyclic or cyclic, aliphatic, aromatic or araliphatic radical having up to 20 carbon atoms, wherein said radical is optionally substituted with up to 5 functional groups and wherein up to 5 of the carbon atoms of said radical is optionally replaced with heteroatoms, and optionally hydrogen when R¹³ and R¹⁵ are remaining substituents; or
R¹⁴ and R¹⁵ together form a divalent, saturated or unsaturated, acyclic or cyclic, aliphatic, aromatic or araliphatic radical having up to 30 carbon atoms, wherein said radical is optionally substituted with up to 5 functional groups and wherein up to 5 of the carbon atoms of said radical is optionally replaced with heteroatoms; and the remaining substituents are each, independently of one another, a saturated or unsaturated, acyclic or cyclic, aliphatic, aromatic or araliphatic radical having up to 20 carbon atoms, wherein said radical is optionally substituted with up to 5 functional groups and wherein up to 5 of the carbon atoms of said radical is optionally replaced with heteroatoms, and R¹³ is optionally hydrogen.

20. The process according to claim 11, wherein said tertiary sp³-hybridized amine or sp²-hybridized imine is trimethylamine, dimethylethylamine, dimethyl-n-propylamine, diethylmethylamine, triethylamine, tri-n-propylamine, di-n-propylmethylamine, tri-n-butylamine, di-n-butylmethylamine, tri-n-pentylamine, N-methylpiperidine, N,N-dimethylaniline, N-methylmorpholine, N-methylimidazole, N-ethylimidazole, N-(1-propyl)imidazole, N-(1-butyl)imidazole, N-(1-hexyl)imidazole, N-(1-octyl)imidazole, N-(1-decyl)imidazole, N-(1-dodecyl)imidazole, N-(1-pentadecyl)imidazole, pyridine, 2-methylpyridine, 3-methylpyridine, 4-methylpyridine, 2,4-dimethylpyridine, 2,6-dimethylpyridine, 2-ethylpyridine, 2,6-diethylpyridine, or N,N,N′,N′,N″-pentamethylguanidine.
